(19) United States
(12) Patent Application Publication
(10) Pub. No.: US 2003/0008362 A1
(43) Pub. Date: Jan. 9, 2003

(54) METHOD TO SEPARATE ETHANOL FROM A SIMULTANEOUS SACCHARIFICATION AND FERMENTATION PROCESS

(76) Inventor: Gene E. Lightner, Federal Way, WA (US)

Correspondence Address: Gene E. Lightner, 706 S.W. 296th St., Federal Way, WA, 98023-3549, US

(21) Appl. No.: 09/901,175

(22) Filed: Jul. 9, 2001

(51) Int. Cl. C12P 7/06; C07C 29/74

(52) U.S. Cl. 435/161; 568/913

(57) ABSTRACT

This is a method to remove ethanol from a simultaneous saccharification and fermentation process (SSF) contained in a vessel. The preferred method employs carbon dioxide, supplied to the vessel, to humidify ethanol and volatile compounds formed by fermentation. Ethanol from the process is transmitted to carbon dioxide to humidify the carbon dioxide. The carbon dioxide containing humidified ethanol and carbon dioxide created by fermentation within the SSF process is removed from the vessel and substantially separated from the ethanol. Carbon dioxide, substantially separated from the ethanol, is recycled to the vessel to humidify additional ethanol from the fermentation process within the vessel.

### BACKGROUND OF THE INVENTION

Throughout the world there is increasing interest in converting renewable biomass to usable products such as ethanol. Conversion of wood to alcohol has been practiced during war time due to a shortage of liquid fuels. Because of poor yields and consumption of chemicals the conversion was found not to be economical for peace time use. Present day interest in hydrolysis of biomass is to provide an alternative fuel source to avoid dependence on unreliable imported petroleum crude oil for liquid fuels. Typical dry biomass composition is: lignin 25%, hemicellulose 25%, amorphous cellulose 10%, and crystalline cellulose 40%

Biomass contains two basic constituents, carbohydrates and lignin. The carbohydrate content of the biomass contains cellulose and hemicellulose. Both cellulose and hemicellulose may be converted to sugars of glucose and xylose. Biomass containing hemicellulose and cellulose does not have pores readily accessible by cellulase. Removal of hemicellulose from a biomass creates pores readily accessible by enzymes including cellulase and a lignocellulose containing lignins and cellulose.

A means of removal of hemicellulose derived from a biomass is described by Grothmann, et al, in U.S. Pat. No. 5,125,977, wherein two stages are employed relying on dilute acid to remove hemicellulose from a biomass and subject lignin to self-condensation to produce a pretreated porous lignocellulose.

Hydrolysis of sterilized cellulose within lignocellulose, obtained from biomass frequently rely on cellulase enzymes for hydrolysis. A procedure related by Wilke, et al, in U.S. Pat. No. 3,972,775, wherein cellulase contained in sugar solution, obtained by hydrolysis, is absorbed by cellulose contained within cellulosic materials. Cellulosic materials containing absorbed cellulase are subjected to “a continuous process for enzymatically converting a cellulosic material to soluble sugars”. Thus absorption of cellulase on cellulose is the means described for hydrolysis to create soluble sugars.

“Simultaneous saccharification and fermentation (SSF) using cellibios fermenting yeast Brettanomyces custersii,” is the title of a process presented within U.S. Pat. No. 5,100,791, by Spindler, et al. This process is achieved within substrates from a biomass to yield ethanol and devoid of a method to remove ethanol en situ to preserve contained enzymes. Consequently the process does not establish removal of ethanol formed within the SSF process. It is therefore an object of this invention to obviate the limitations or disadvantages of the prior art.

The present object of this invention is to remove ethanol and volatile compounds created by fermentation within the SSF process from cellulose contained within lignocellulose.

A distinct object of this invention is to substantially contain enzymes in the locality required to continue fermentation within the SSF process.

An object of this invention is to add gaseous carbon dioxide to the SSF process to humidify ethanol and volatile compounds and remove humidified carbon dioxide from the SSF process while removing carbon dioxide created by fermentation within the SSF process.

Another object of this invention is to prevent concentration of ethanol within the SSF process from reaching a concentration of ethanol lethal to the enzymes and yeasts within the SSF process.

Still another object of this invention is to substantially contain sugars within the SSF process to subject the unfermented sugars to further fermentation.

An additional object of this invention is to substantially establish a temperature and maintain substantially isothermal conditions within the SSF process.

A supplementary object of this invention is means for removing ethanol from humidified carbon dioxide, wherein the humidified carbon dioxide forms condensed ethanol, which may require use of a demister, to remove ethanol separated from carbon dioxide containing ethanol.

With the above and other objects in view, this invention relates to the novel features and alternatives and combinations presently described in the brief description of the invention.

### PRINCIPLES APPLIED BY THE INVENTION

The principles applied herein employ Dalton's law and Raoult's law. Dalton's law of partial pressure may be expressed mathematically as P=p_{A}+p_{B} where p_{A} and p_{B} are the partial pressures of vapors A and B respectively and P is the total pressure.

For only A and B, P=p_{A}+p_{B}, and the mole ratio of B to A is p_{B}/p_{A}=p_{B}/P−p_{B}.

The weight ratio of A/B is p_{B}/P−p_{B} (molecular weight of B)/(average molecular weight of P−p_{B}). This is the equation used for humidity calculations when A is a gas and B is the vapor humidified.

Raoult's law of partial pressure may be expressed mathematically as pₛₒₗᵥₑₙₜ=p°ₛₒₗᵥₑₙₜ×Nₛₒₗᵥₑₙₜ where pₛₒₗᵥₑₙₜ is the partial vapor pressure of the solvent, p°ₛₒₗᵥₑₙₜ is the vapor pressure of the solvent times the mole fraction, N, of the solvent in a solution. Applying Raoult's law, let N=0.1 (the mole fraction of ethanol in a fermentation broth) and p°ₑₜₕₐₙₒₗ at a temperature Of 100° F. the partial vapor pressure=2.5 psia, then pₑₜₕₐₙₒₗ=0.1×2.5 psia=0.25 psia.

The molecular weight of ethanol=46 and carbon dioxide has a molecular weight=44.

Applying the equation used for humidity, and making P=15 psia for a total pressure of humidified carbon dioxide, the weight ratio of ethanol/carbon dioxide is 0.25/15−0.25×46/44=0.018 lb. of ethanol /lb. of carbon dioxide.

Thus a fermentation process can have ethanol removed by co-mingling carbon dioxide with the fermentation process to form carbon dioxide humidified with ethanol. The carbon dioxide humidified with ethanol can also contain other humidified compounds.

Raoult's law predicts that any volatile compound will form a partial vapor pressure of the volatile compound depending or the vapor pressure and mole fraction of the volatile compound in the fermentation process. The equation used for humidity asserts that when a gas is humidified, the humidified gas may contain any partial vapor pressure of a volatile compound. Thus, if the humidified carbon dioxide contains a partial vapor pressure of a volatile compound contained in the fermentation broth of the same partial vapor pressure of the same volatile compound then further humidification of the volatile compound will not occur. The same temperature of fermentation both and the humidified carbon dioxide is assumed. For additional information, review F. Daniels, Outlines of Physical Chemistry and G. G. Brown et al., Unit Operations.

Hemicellulose within a biomass is removed to create a lignocellulose containing lignins and cellulose and in the process creates pores readily accessible by enzymes including cellulase. A means of removal of lignocellulose from a biomass is described by Grothmann, et al, in U.S. Pat. No. 5,125,977, wherein two stages are employed relying on dilute acid to remove hemicellulose from a biomass and subject lignin to self-condensation to produce a treated porous lignocellulose. Thus a porous lignocellulose is created by removal of hemicellulose from a biomass using a dilute acid.

Cellulase enzymes used for hydrolysis of cellulose contained in lignocellulose include endoglucanases which act randomly on cellulose and exoglucanases which include glucanhydrolases for liberation of glucose from the end of a cellulose chain and cellobiohydrolases that liberate cellobiose from the end of a cellulose chain. Also included is β-glucosiddases for liberation of glucose from cellobiose and cellodextrins. Therefore the classes of enzymes work synergistically to hydrolyze cellulose to form water soluble sugars as reported in Enzymatic Hydrolysis on the internet. Water soluble sugars are then subjected to fermentation by microorganisms to produce ethanol and carbon dioxide. Fermentation takes place in which ethanol and carbon dioxide are produced from the group of carbohydrates which include glucose and cellobiose including an individual or a combination of these thereof. The simultaneous saccharification and fermentation process is established at a level of nutrients used for fermentation.

### BRIEF DESCRIPTION OF THE INVENTION

The present invention in its broadest aspect, comprises a method to remove ethanol from fermentation contained in a simultaneous saccharification and fermentation (SSF) process contained in a vessel. The preferred method employs carbon dioxide, supplied to the vessel, to humidify ethanol and volatile compounds formed by fermentation. By combining the carbon dioxide, produced by fermentation with carbon dioxide humidified by ethanol, within the SSF process humidified carbon dioxide is formed. Carbon dioxide humidified by ethanol is removed from the vessel and substantially separated from the ethanol. The carbon dioxide, substantially separated from the ethanol, is then recycled to humidify additional ethanol from the fermentation process within the vessel. Lignocellulose, containing cellulose, supplied to the SSF vessel is subjected to hydrolysis to form sugars for fermentation. Hydrolysis residue containing lignins from the SSF vessel is subjected to filtration to produce a filtrate. The filtrate is recycled to the SSF vessel. The filtered residue is extracted by water to produce extracted residue for subsequent disposal and an extractate subject to additional treatment.

Characteristics of the invention include;

By providing the SSF vessel with a lignocellulose, cellulose is subjected to hydrolysis.

Removing carbon dioxide, provided to the SSF vessel, humidified by ethanol and other volatile components contained in the SSF vessel is accomplished.

Humidified carbon dioxide, containing carbon dioxide created by fermentation, is removed from the SSF vessel.

Humidified carbon dioxide, containing ethanol and other volatile components, is separated to provide a liquid containing ethanol and carbon dioxide containing ethanol.

Sugars to form ethanol and carbon dioxide by fermentation, within the SSF vessel, create ethanol and volatile components. Volatile components contained in the humidified carbon dioxide can be of several types including aldehydes, alcohols, esters and acids.

Fermentation produces heat, that is substantially proportional to ethanol produced, which is removed from the fermentation vessel by vaporization of ethanol and other volatiles contained in the humidified carbon dioxide to control temperature of the SSF process.

Enzymes fundamental to the SSF process are located and contained within the SSF vessel. Temperature is maintained at substantially isothermal conditions and pH level is maintained within the SSF process.

### BRIEF DESCRIPTION OF THE DRAWINGS

The features that are considered characteristic of this invention are set forth in the appended claims. This invention, however, both as to its origination and method of operations as well as additional advantages will best be understood from the following description when read in conjunction with the accompanying drawings in which:

FIG. 1 is a flow sheet denoting the invention as set forth in the appended claims.

FIG. 2 is a flow sheet denoting a method for substantially separating ethanol from carbon dioxide containing ethanol.

FIG. 3 is a flow sheet denoting a method to produce a porous lignocellulose from a biomass.

### DETAILED DESCRIPTION OF THE PREFERRED EMBODIMENT

In the preferred embodiment of the present invention, carbon dioxide is added to a simultaneous saccharification and fermentation (SSF) vessel, for humidifying the carbon dioxide with ethanol within the SSF process. The predetermined operating level of temperature range within the SSF process is about 30° C. to about 40° C.

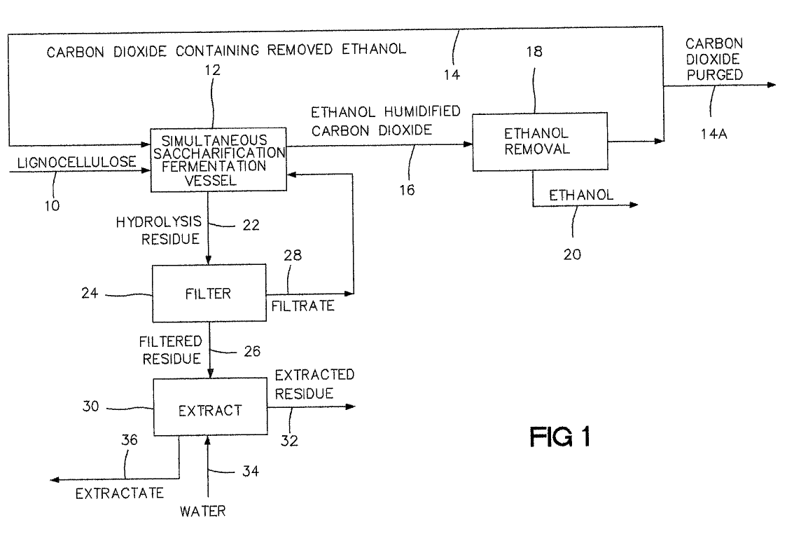

The flow diagram of FIG. 1 illustrates the general preferred embodiment of the present invention. In the diagram, rectangles represent stages, operations or functions of the present invention and not necessarily separate components. Arrows indicate direction of flow of material in the method.

Referring to FIG. 1, lignocellulose 10 is conveyed to simultaneous saccharification and fermentation vessel 12 and added, within the vessel, carbon dioxide containing removed ethanol 14, subjected to simultaneous hydrolysis, saccharification, and fermentation to form carbon dioxide and ethanol by fermentation. The ethanol humidified by carbon dioxide combined with carbon dioxide formed by fermentation is separated from the simultaneous saccharification and fermentation vessel 12 to form ethanol humidified carbon dioxide 16 and conveyed to ethanol removal stage 18 to remove ethanol 20 and form carbon dioxide containing removed ethanol 14 for recycle. Hydrolysis residue 22 from simultaneous saccharification and fermentation vessel 12 is conveyed to filter stage 24 and subjected to fixation to produce filtrate 28 and filtered residue 26 of solids, conveyed to extract stage 30. Filtrate 28 is recycled and conveyed to simultaneous saccharification and fermentation vessel 12. Residue 26 is extracted by water 34 to produce an aqueous extractate 36 and extracted residue 32. Extractate 36 is subject to additional treatment. Hydrolysis of cellulose contained in lignocellulose is achieved by cellulase type enzymes to yield water soluble carbohydrates which are then fermented to form ethanol and carbon dioxide. Porous lignocellulose, containing cellulose 10, renders cellulose readily accessible to cellulase type enzymes contained within the simultaneous saccharification and fermentation vessel 12. The method described in FIG. 1 employs humidification for transmitting ethanol from fermentation to insoluble carbon dioxide contained within carbon dioxide containing removed ethanol 14. The method will purge 14A from carbon dioxide containing removed ethanol 14 and is available for further treatment to form ethanol distillate and gaseous carbon dioxide. Humidified carbon dioxide 16 may contain various volatile compounds from the fermentation. The method can be operated by continuous operation. The method can be pressurized and operated to substantially adjust the pressure on the carbon dioxide to control the pressure within the fermentation vessel. Carbohydrates, capable of fermentation to produce ethanol and carbon dioxide, consist of the group which include glucose and cellobiose including an individual or a combination of these thereof.

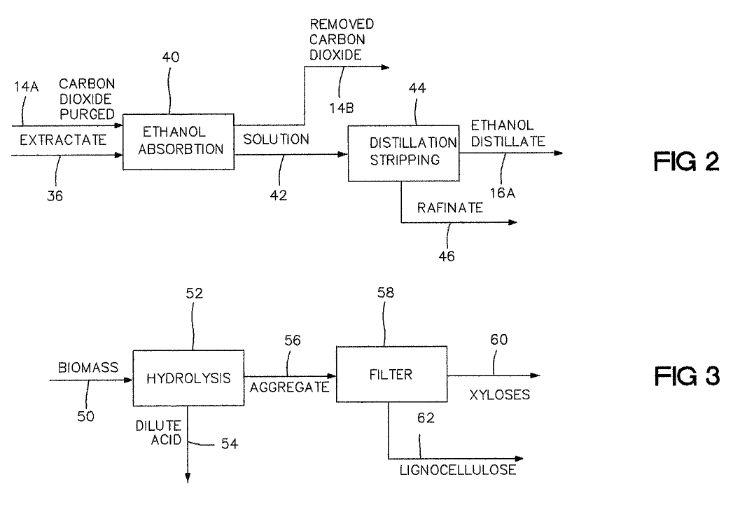

Referring to FIG. 2, The purged humidified carbon dioxide 14A is added to extractate 36 and transmitted to ethanol absorption stage 40 and absorbed of ethanol to produce solution 42 and removed gaseous carbon dioxide 14B. Solution 42 is transmitted to distillation stripping stage 44 and distilled to form ethanol distillate 16A and raffinate 46.

Referring to FIG. 3, biomass 50 is transported to hydrolysis stage 52, combined with dilute acid 54 to attain hydrolysis of hemicellulose, contained within biomass, 50. Aggregate of solids and sugars 56, formed by hydrolysis, is transported to filter stage 58. Filter stage 58 performs filtration to form lignocellulose 62 and xyloses 60. Hemicellulose, contained in biomass, is accordingly hydrolyzed to produce lignocellulose 62 substantially devoid of hemicellulose. Lignocellulose 62, substantially devoid of hemicellulose, is accordingly rendered porous to improve absorption of enzymes by cellulose contained within lignocellulose. Acid within lignocellulose 62 and xyloses 60 is substantially neutralized. Xyloses 60, when neutralized, are subjected to fermentation to form ethanol. Hemicellulose upon hydrolysis forms xyloses and glucose. Xyloses 60 containing glucose is also subjected to fermentation to form ethanol. Hydrolysis stage 52 may be configured as two individual stages to provide relative ease and relative difficulty of hemicellulose hydrolysis to prevent or limit formation of furfural.

What is claimed is:

1. A method to separate ethanol from a simultaneous saccharification and fermentation process contained in a vessel, which comprises:
providing a simultaneous saccharification fermentation vessel in which ethanol and carbon dioxide are produced by fermentation, and
providing gaseous carbon dioxide to said vessel to humidify said ethanol within said vessel by said carbon dioxide, and
combining said carbon dioxide, produced by fermentation, with the humidified gaseous carbon dioxide to form humidified carbon dioxide, and
separating the resulting humidified carbon dioxide from the fermentation vessel, and
removing means for removing ethanol from said humidified carbon dioxide to supply carbon dioxide containing ethanol for recycle, and purged carbon dioxide containing ethanol to remove carbon dioxide produced by fermentation, thereby removing ethanol from the humidified carbon dioxide to produce ethanol and carbon dioxide containing ethanol for recycle and removing carbon dioxide produced by fermentation.

2. The method of claim 1 where said simultaneous saccharification and fermentation process is established and maintained at a predetermined ethanol level.

3. The method of claim 1 wherein said simultaneous saccharification and fermentation process is established at a predetermined temperature and maintained at substantially isothermal conditions.

4. The method of claim 1 wherein said simultaneous saccharification and fermentation process, fermentation takes place in which ethanol and carbon dioxide are produced from the group of carbohydrates which include glucose and cellobiose including an individual or a combination of these thereof.

5. The method of claim 1 wherein said simultaneous saccharification and fermentation process, contained in a vessel, is established at a predetermined level of pH within said vessel and substantially maintained at the pH level.

6. The method of claim 1 wherein said simultaneous saccharification and fermentation process is established at a predetermined level of nutrients used for fermentation.

7. The method of claim 1 wherein said simultaneous saccharification and fermentation process is provided with sterile lignocellulose to maintain fermentation.

8. The method of claim 1 wherein said simultaneous saccharification and fermentation process is provided with porous lignocellulose, substantially devoid of hemicellulose derived from biomass, to maintain fermentation.

9. The method of claim 7 wherein said lignocellulose, subjected to saccharification and fermentation, form a residue containing lignins, and is removed from said vessel

10. The method of claim 8 wherein said residue, containing lignins, is subjected to filtration to produce a filtrate and filtered residue.

11. The method of claim 9 wherein said filtered residue is subjected to extraction by water to produce water extracted residue and an aqueous extractate.

12. The method of claim 1 wherein said humidified carbon dioxide is absorbed by an aqueous extractate to produce gaseous carbon dioxide and a solution containing ethanol.

13. The method of claim 12 wherein said solution containing ethanol is subjected to distillation to produce a distillate containing ethanol and produce a raffinate substantially devoid of ethanol.

14. The method of claim 1 wherein said removing means for removing ethanol from said humidified carbon dioxide, said humidified carbon dioxide is subjected to cooling, to produce condensed ethanol separated from carbon dioxide containing ethanol for recycle.

15. The method of claim 1 wherein said removing means for removing ethanol from said humidified carbon dioxide, said humidified carbon dioxide is subjected to pressurization, to produce condensed ethanol separated from carbon dioxide containing ethanol for recycle.

16. The method of claim 1 wherein the purged carbon dioxide containing ethanol is subjected to absorption by the aqueous extractate to provide a solution containing ethanol and removed carbon dioxide, produced by fermentation, substantially devoid of ethanol.

17. The method of claim 16 wherein the solution containing ethanol is subjected to distillation to produce an ethanol distillate and a rafinate.
